(19) United States
(12) Patent Application Publication
(10) Pub. No.: US 2022/0401037 A1
(43) Pub. Date: Dec. 22, 2022

(54) ML-BASED ANOMALY DETECTION AND DESCRIPTIVE ROOT-CAUSE ANALYSIS FOR BIODATA

(71) Applicant: BIOINTELLISENSE, INC., Redwood City, CA (US)

(72) Inventors: Mehdi Sadeghzadeh, Thornhill (CA); David Jonq Wang, Palo Alto, CA (US); Nivedita Khobragade, San Mateo, CA (US); Zongde Qiu, Cupertino, CA (US); I-Ting Chen, San Jose, CA (US); Christopher Charles Reynolds, San Jose, CA (US); Alexander Katsis, San Mateo, CA (US); James R. Mault, Evergreen, CO (US)

(21) Appl. No.: 17/349,166

(22) Filed: Jun. 16, 2021

(51) Int. Cl. A61B 5/00; G16H 50/20; G16H 50/30

(52) U.S. Cl. CPC A61B 5/7267; A61B 5/7275; A61B 5/7246; A61B 5/7282; A61B 5/725; G16H 50/20; G16H 50/30

(57) ABSTRACT

In an example, a method includes collecting biodata of a subject. The method includes generating or updating a personalized ML model of the subject from the biodata of the subject. The method includes detecting anomalies in the biodata based on the personalized ML model. The method includes filtering the detected anomalies to determine whether the detected anomalies indicate that the subject has a clinical condition or is at risk of having the clinical condition.

### FIELD

The embodiments discussed herein are related to machine-learning (ML)-based anomaly detection and descriptive root-cause analysis for biodata.

### BACKGROUND

Unless otherwise indicated herein, the materials described herein are not prior art to the claims in the present application and are not admitted to be prior art by inclusion in this section.

Smartphones, wearable devices (such as smart watches and fitness trackers), and other personal electronic devices and sensor devices are becoming more widely used and make it possible to collect biodata of users regularly. Many users use this information to monitor various aspects of their health, such as heart rate, calories burned, and hours of sleep. Some embodiments herein relate to various applications for such biodata.

The subject matter claimed herein is not limited to implementations that solve any disadvantages or that operate only in environments such as those described above. Rather, this background is only provided to illustrate one example technology area where some implementations described herein may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

In an example embodiment, a method includes collecting biodata of a subject. The method includes generating or updating a personalized ML model of the subject from the biodata of the subject. The method includes detecting anomalies in the biodata based on the personalized ML model The method includes filtering the detected anomalies to determine whether the detected anomalies indicate that the subject has a clinical condition or is at risk of having the clinical condition.

In another example embodiment, a non-transitory computer-readable storage medium has computer-readable instructions stored thereon that are executable by a processor device to perform or control performance of operations. The operations include collecting biodata of a subject. The operations include generating or updating a personalized ML model of the subject from the biodata of the subject. The operations include detecting anomalies in the biodata based on the personalized ML model. The operations include filtering the detected anomalies to determine whether the detected anomalies indicate that the subject has a clinical condition or is at risk of having the clinical condition.

In another example embodiment, a method includes collecting a first time series of measurements of a first biological parameter of a subject. The method includes collecting a second time series of measurements of a second biological parameter of a subject. The method includes generating or updating a personalized ML model of the subject from the first time series and the second time series. The method includes detecting a first anomaly in the first time series of measurements of the first biological parameter based on the personalized ML model. The method includes detecting a second anomaly in the second time series of measurements of the second biological parameter based on the personalized ML model. The method includes, in response to the first and second anomalies satisfying filter criteria and timings of the first and second anomalies having a correlation that satisfies a correlation condition, determining that the anomalies indicate the subject has a clinical condition or is at risk of having the clinical condition.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates an example operating environment;

FIGS. 2A and 2B illustrate an example sensor device that may be implemented in the environment of FIG. 1;

FIG. 3 is a perspective view of another example sensor device that may be implemented in the environment of FIG. 1;

FIG. 4 illustrates an example computing device that may be implemented in the environment of FIG. 1;

FIG. 5 illustrates experimental results for a subject;

FIGS. 6A and 6B illustrate an example descriptive root-cause analysis graph for the experimental results of FIG. 5; and

FIG. 7 is a flowchart of a method to detect anomalies, all arranged in accordance with at least one embodiment described herein.

### DETAILED DESCRIPTION OF SOME EXAMPLE EMBODIMENTS

Some embodiments herein detect anomalies in biodata automatically using adaptive artificial intelligence (AI)/ML algorithms. The biodata may be collected by devices such as BIOINTELLISENSE'S BIOSTICKER and/or BIOBUTTON sensor devices and/or other sensors or sensor devices. The algorithms may be configured to discover anomalies without human intervention in a continuous personalized health monitoring platform of vital signs, physiological biometrics, behavioral patterns, and/or symptomatic events, collectively referred to herein as biodata. The ML algorithms may be executed periodically (e.g., every 2 hours) and may identify anomaly points in, e.g., a subject's most recent biodata according to a personalized ML model generated from collected biodata of the subject in the past (e.g., past 24 hours).

Examples of biological parameters that may be measured and included in the biodata may include heart rate, respiratory rate, skin temperature, ambient temperature, motion, and/or others.

After detection of anomalies, additional processing may be performed to trigger alerts according to, e.g., the detected anomaly points' counts and/or distribution in one or more determined rolling time windows and/or other criteria (e.g., other input feature conditions and values).

Some embodiments may additionally include or provide a descriptive root-cause analysis tool for detected anomalies. This tool may provide easy-to-understand output graphs and statements that demonstrate relevance or importance of input features and values for each detected anomaly which may be helpful for disease diagnostics and further actions plan.

Embodiments herein may be implemented as a software package to integrate to a data service and monitoring platform that may be used for one or more of the following applications:
Respiratory illness (e.g., sleep apnea, asthma, etc.) alerts
Covid-19-like symptoms alerts
Infection-like symptom alerts
Mental health deterioration or improvements: Depression, Anxiety, ADHD, Insomnia
Substance Abuse detection and cessation alerts
Post vaccine effects analysis
Body temperature estimation and fever alerts
Pre-surgical baseline
Pre-operative anesthesia
In-home condition management
Chronic and complex care management
Post-surgical recovery & rehabilitation
Post-hospital virtual care
Cardiac Arrythmia screening and diagnostics
Fall Risk alerts based on gait analysis and other behavioral patterns

Embodiments herein may include personalized health monitoring solutions as the AI/ML algorithms may be executed and/or adapted using most recent plus past individual biodata and is not limited to pre-trained models with population-based data. In some embodiments, an initial ML model of a subject may be generated using or from population-based data and the initial ML model may be personalized and adapted incrementally each time new biodata of the subject is collected.

Some embodiments herein implement adaptive ML algorithms. In contrast to traditional ML algorithms which typically require a significant amount of time to adapt to changes, adaptive ML algorithms can quickly adapt to changes. By using adaptive ML algorithms, each time that a new set of biodata of a subject is collected, the new set of biodata may be processed by the adaptive ML algorithms to update the subject's personalized ML model and the new set of biodata may be analyzed for anomalies based on the updated personalized ML model. This is an example of incremental learning.

Embodiments of the personalization methodology described herein may be more effective in comparison with traditional approaches in which variations in input data from a population-based baseline are considered. Some embodiments may be more effective because each subject's model may be personalized and adapted/updated most recent individual status and conditions for each run. The ML algorithms may be executed periodically (e.g., every 2 hours) and may use previously collected individual data for updates, adaption, and anomaly (e.g., outlier) detection.

Embodiments herein may implement supervised ML and/or unsupervised ML. Alternatively or additionally, embodiments herein may be implemented in situations in which training data with reliable “ground truth” is unavailable, contrary to supervised ML techniques in which a reliable “ground truth” is required. Some embodiments herein may implement incremental learning in which input data is used, e.g., continuously, to further train the ML model.

In terms of processing time, each time a personalized ML model is updated in view of a subject's most recent biodata, it may take approximately 10 seconds to update for 24 hours of biodata, 23 seconds to update for several days of biodata, or some other amount of execution time for the same or different amounts time coverage of the biodata. According to embodiments herein, training and testing phases need not be separated nor is a long training time required, contrary to deep learning algorithms which may require a long training time.

Some embodiments herein may be generally applied for screening and/or diagnostic purposes, e.g., to determine whether a subject is at risk of having a clinical condition or whether the subject has the clinical condition. Accordingly, some embodiments may detect various clinical conditions or risk of having various clinical conditions, such as fevers, infections, vaccine reactions, or the like, according to a combination of detected anomalies. Insofar as different clinical conditions may present with or be indicated by different combinations of anomalous biodata, the combinations of anomalies detected in the biodata may be used to at least tentatively identify or determine one or more clinical conditions or risk thereof of a subject at any given time.

Reference will now be made to the drawings to describe various aspects of example embodiments of the invention. It is to be understood that the drawings are diagrammatic and schematic representations of such example embodiments, and are not limiting of the present invention, nor are they necessarily drawn to scale.

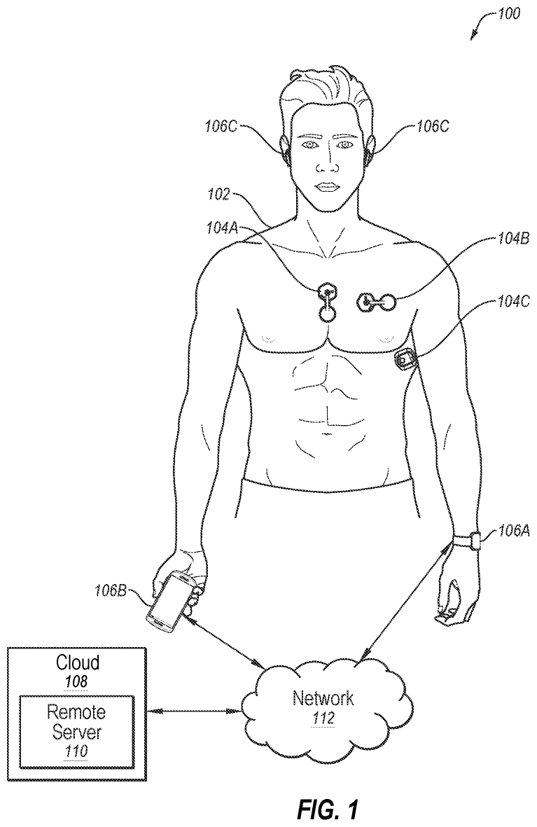

FIG. 1 illustrates an example operating environment 100 (hereinafter “environment 100”), arranged in accordance with at least one embodiment described herein. The environment 100 includes a subject 102 with one or more sensor devices 104A, 104B, 104C (hereinafter collectively “sensor devices 104” or generically “sensor device 104”) and/or one or more personal electronic devices 106A, 106B, 106C (hereinafter collectively “personal electronic devices 106” or generically “personal electronic device 106”). The environment 100 may additionally include a cloud computing environment (hereinafter “cloud 108”) that includes at least one remote server 110 and a network 112.

The sensor devices 104 may be implemented as sensor patches or stickers that attach directly to skin of the subject 102. The personal electronic devices 106 may each include a desktop computer, a laptop computer, a tablet computer, a smartphone, a wearable electronic device (e.g., smart watch, activity tracker, headphones, ear buds, etc.), or other personal electronic device. In the illustrated example, the personal electronic device 106A may include a smart watch, the personal electronic device 106B may include a smartphone, and the personal electronic device 106C may include a pair of ear buds.

Each of the sensor devices 104 and/or the personal electronic devices 106 may include one or more sensors to generate biodata of the subject 102. The biodata may be in the form of a time series of measurements of one or more physiological parameters (e.g., vital signs, biometrics); a time series of measurements of one or more other parameters that influence the physiology of the subject 102; a time series of measurements of one or more behavioral patterns; symptomatic events (e.g., coughing, sneezing, vomiting, limping, falling) detected in one or more of the time series of measurements; and/or other measurements or parameters. The physiological parameters, the other parameters that influence the physiology of the subject 102, behavioral parameters, and the symptomatic events are collectively referred to herein as biological parameters. The biological parameters may include heart rate of the subject 102, blood pressure of the subject 102, respiratory rate of the subject 102, skin temperature of the subject 102, heart rate variability of the subject 102, respiratory rate variability of the subject 102, ambient temperature around the subject 102, motion vector values (e.g., acceleration as measured by an accelerometer or other motion sensor) of the subject 102, or other biological parameter. Alternatively or additionally, the biological parameters may include core body temperature of the subject 102, blood oxygenation of the subject 102, blood flow of the subject 102, electrical activity of the heart of the subject 102, electrodermal activity (EDA) of the subject 102, sound around the subject 102, EEG brain waves of the subject 102, light level or UV light level of an environment of the subject 102, coughing, sneezing, vomiting, asthma attack, apnea, hypopnea, arrhythmias, or other biological parameters. In some embodiments, the biological parameters may include sleep pattern (e.g., when the user sleeps), sleep position, body position (e.g., incline angle), gait, or the like.

In some embodiments, the biodata may include subject-reported data. For example, when a subject experiences a given symptom, the subject may operate one or more of the personal electronic devices 106 or other devices in the environment 100 to record the occurrence of the given symptom. Some example symptoms that may be recorded by the user include heart palpitations, feeling a pause between heartbeats, lightheadedness, passing out, shortness of breath, chest pain, nausea, vomiting, coughing, sneezing, choking, falling, or other symptoms. In some embodiments, the subject-reported data may be used to label other biodata, e.g., for training purposes. For example, if a subject reports vomiting, features in audio data generated by a microphone and/or motion data generated by an accelerometer that occur in the respective data at about the same time (or shortly before) the subject-reported vomiting symptom may be labeled as being indicative of the vomiting symptom. The labeled data may be used as training data in generating or updating the subject's personalized ML model.

The network 112 may include one or more wide area networks (WANs) and/or local area networks (LANs) that enable the sensor devices 104, the personal electronic devices 106, the cloud 108, and/or the remote server 110 to communicate with each other. In some embodiments, the network 112 includes the Internet, including a global internetwork formed by logical and physical connections between multiple WANs and/or LANs. Alternately or additionally, the network 112 may include one or more cellular radio frequency (RF) networks and/or one or more wired and/or wireless networks such as 802.xx networks, BLUETOOTH access points, wireless access points, IP-based networks, or other suitable networks. The network 112 may also include servers that enable one type of network to interface with another type of network.

All of the sensors in the environment 100 to generate biodata of the subject 102 may be included in a single device, such as one of the sensor devices 104 or personal electronic devices 106. Alternatively or additionally, the sensors in the environment 100 to generate biodata of the subject 102 may be distributed between two or more devices. The remote server 110 and the sensor devices 104 or other sensors in the environment 100 may be owned by, manufactured by, sold by, provided by, under the control of, and/or associated with the same entity or service provider. For example, the remote server 110 may be under the control of the same company that sells the sensor devices 104 or other sensors in the environment 100. Alternatively or additionally, one or more of the sensor devices 104 or other sensors in the environment 100 may be third-party sensors or sensor devices. For example, one or more of the sensor devices 104 or other sensors in the environment 100 may be manufactured and/or sold by a different entity than the entity that controls the remote server 110. In these and other embodiments, the remote server 110 or other devices in the environment 100 may include one or more plugins or software interfaces to collect biodata from third-party sensors or sensor devices, which biodata may be used as described herein.

Each sensor may include any of a discrete microphone, an accelerometer, a gyrometer sensor, a blood pressure sensor, an optical spectrometer sensor, an electro-chemical sensor, a thermometer, an oxygen saturation sensor, a photoplethysmography (PPG) sensor, an optical sensor, a heart rate sensor, an electrocardiogram (ECG or EKG) sensor, a peripheral oxygen saturation (SpO₂) sensor (also known as a pulse oximeter), EDA sensor, or other sensor. In some embodiments, distributing the sensors between two or more sensor devices 104 or personal electronic devices 106 at different locations on the subject 102 may be beneficial for a more robust set of data to analyze the subject 102. For example, different locations of the sensors may measure different biological parameters based on their respective locations proximate different parts of the anatomy of the subject 102. Each sensor may be configured to generate a data signal, e.g., a time series of measurements, of, e.g., heart rate, respiratory rate, skin temperature, ambient temperature, motion, or other biological parameter(s) of the subject 102.

In some embodiments, one of the personal electronic devices 106, such as the smartphone 106B or a desktop or laptop computer, may collect data signals or data derived therefrom from the sensor devices 104 and/or the other personal electronic devices 106. The smartphone 106B may apply AI, ML, and/or other processing to the data signals, portions thereof, or data derived from the data signals and sent to the smartphone 106B, to generate and/or update a personalized ML model of the subject 102, detect anomalies in the data signals (or more generally in the biodata), filter detected anomalies, and/or generate alerts.

Alternatively or additionally, the data signals generated by the sensors in the sensor devices 104 and the personal electronic devices 106 and/or data derived therefrom may be uploaded, e.g., periodically, by the corresponding sensor device 104 or personal electronic device 106 to the remote server 110. In some embodiments, one or more of the sensor devices 104 or personal electronic devices 106 or another device may act as a hub that collects data signals or data derived therefrom from other sensor devices 104 and/or personal electronic devices 106 and uploads the data signals or data derived therefrom to the remote server 110. For example, the hub may collect data over a local communication scheme (WI-FI, BLUETOOTH, near-field communications (NFC), etc.) and may transmit the data to the remote server 110. In some embodiments, the hub may act to collect the data and periodically provide the data to the remote server 110, such as once per week. An example hub and associated methods and devices are disclosed in U.S. Pat. No. 10,743,091, which is incorporated herein by reference.

The remote server 110 may include a collection of computing resources available in the cloud 108. The remote server 110 may be configured to receive data signals and/or data derived from data signals collected by one or more sensors or other devices, such as the sensor devices 104 and the personal electronic devices 106 within the environment 100. Alternatively or additionally, the remote server 110 may be configured to receive from the sensors (e.g., directly or indirectly via a hub device) relatively small portions of the data signals, or even larger portions or all of the data signals. The remote server 110 may apply AI, ML, and/or other processing to the data signals, portions thereof, or data derived from the data signals and sent to the remote server 110, to generate and/or update a personalized ML model of the subject 102, detect anomalies in the data signals (or more generally in the biodata), filter detected anomalies, and/or generate alerts.

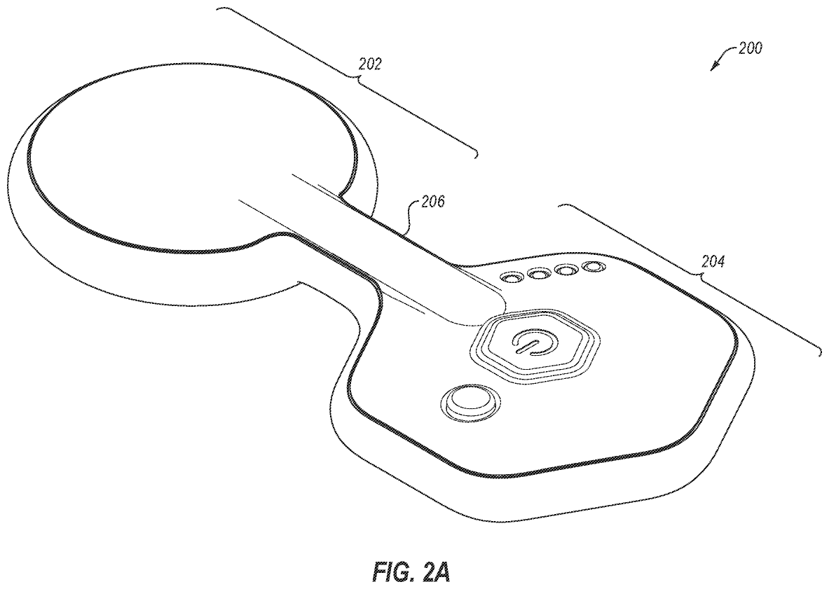

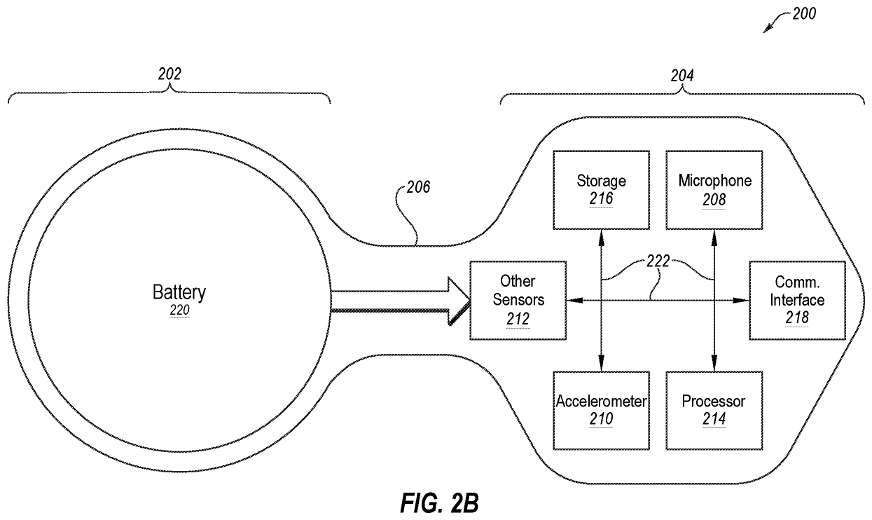

FIGS. 2A and 2B illustrate an example sensor device 200 that may be implemented in the environment of FIG. 1, arranged in accordance with at least one embodiment described herein. FIG. 2A is a perspective view and FIG. 2B is a block diagram of the sensor device 200. The sensor device 200 may include, be included in, or correspond to any of the sensor devices 104 of FIG. 1, such as the sensor devices 104A and 104B of FIG. 1. The sensor device 200 may be configured to monitor one or more biological parameters of the subject 102 while disposed, e.g., on the body of the subject 102. For example, the sensor device 200 may generate one or more data signals of one or more biological parameters of the subject 102.

The sensor device 200 may include a first lobe 202 and a second lobe 204 connected by a band 206. In some embodiments, the first lobe 202 may be narrower than the second lobe 204. The first lobe 202 may have a generally circular shape and the second lobe 204 may have a generally hexagonal shape, although any shape may be used. The sensor device 200 may include a microphone 208, an accelerometer 210, other sensors 212, a processor 214, a storage 216, a communication interface 218, a battery 220, and a communication bus 222.

The microphone 208 may be used to record sound and may be oriented to face the skin of the subject 102. While the term microphone is used, it will be appreciated that term includes any type of acoustic sensor that may be configured to detect sound waves and convert them into a readable signal such as an electronic signal. For example, a piezoelectric transducer, a condenser microphone, a moving-coil microphone, a fiber optic microphone, a MicroElectrical-Mechanical System (MEMS) microphone, etc. or any other transducer may be used to implement the microphone 208.

The accelerometer 210 may be used to measure acceleration of at least a portion of the subject 102, such as the chest of the subject 102, based on the sensor device 200 being adhered to the portion of the subject 102.

The other sensors 212 may include any number of other sensors such as a gyrometer sensor, a blood pressure sensor, an optical spectrometer sensor, an electro-chemical sensor, an oxygen saturation sensor, a thermometer, a PPG sensor, an ECG sensor, an EDA sensor, etc. or any combinations thereof. Any other of a variety of sensors may also be associated with the sensor device 200. A gyrometer sensor may be used to measure angular velocity of at least a portion of the subject 102, such as the chest of subject 102. An oxygen saturation sensor may be used to record blood oxygenation of the subject 102 to generate a blood oxygenation level signal of the subject 102. A thermometer may be used to record temperatures associated with the subject 102, including skin temperature and/or core body temperature. A PPG sensor may be used to record blood flow of the subject 102. An ECG sensor may be used to measure electrical activity of the heart of the subject 102 to determine the heart rate of the subject 102 and/or other parameters. An EDA sensor may be used to measure EDA of the skin of the subject 102. A volatile organic compound (VOC) detector may be used to detect various organic molecules that may be coming off of the subject 102 or their sweat. An optical sensor may be used to monitor or detect changes in color, such as changes in skin coloration of the subject 102. Alternatively or additionally, a spectrometer may measure electromagnetic (EM) radiation and may be configured to detect variations in reflected EM radiation. For example, such a sensor may detect changes in color in a molecule exposed to multi-spectral light (e.g., white light), and/or may detect other changes in reflected EM radiation outside of the visible spectrum (e.g., interaction with ultra-violet rays, etc.).

The processor 214 may include any device or component configured to monitor and/or control operation of the sensor device 200. For example, the processor 214 may retrieve instructions from the storage 216 and execute those instructions. As another example, the processor 214 may read the signals generated by the sensors (e.g., the microphone 208, the accelerometer 210, and/or the other sensors 212) and may store the readings in the storage 216 or instruct the communication interface 218 to send the readings to another electronic device, such as the remote server 110 of FIG. 1. In some embodiments, the processor 214 may include an arithmetic logic unit, a microprocessor, a general-purpose controller, or some other processor or array of processors, to perform or control performance of operations as described herein. The processor 214 may be configured to process data signals and may include various computing architectures including a complex instruction set computer (CISC) architecture, a reduced instruction set computer (RISC) architecture, or an architecture implementing a combination of instruction sets. Although illustrated as a single processor 214, multiple processor devices may be included and other processors and physical configurations may be possible. The processor 214 may be configured to process any suitable number format including, but not limited to two's compliment numbers, integers, fixed binary point numbers, and/or floating point numbers, etc. all of which may be signed or unsigned. In some embodiments, the processor 214 may perform processing on the readings from the sensors prior to storing and/or communicating the readings. For example, raw analog data signals generated by the microphone 208, the accelerometer 210, and/or the other sensors 212 may be downsampled, may be converted to digital data signals, and/or may be processed in some other manner.

The storage 216 may include non-transitory computer-readable storage media or one or more non-transitory computer-readable storage mediums for carrying or having computer-executable instructions or data structures stored thereon. Such non-transitory computer-readable storage media may be any available non-transitory media that may be accessed by a general-purpose or special-purpose computer, such as the processor 214. By way of example such non-transitory computer-readable storage media may include Random Access Memory (RAM), Read-Only Memory (ROM), Electrically Erasable Programmable Read-Only Memory (EEPROM), flash memory devices (e.g., solid state memory devices), or any other non-transitory storage medium which may be used to carry or store desired program code in the form of computer-executable instructions or data structures and which may be accessed by a general-purpose or special-purpose computer. In some embodiments, the storage 216 may alternatively or additionally include volatile memory, such as a dynamic random access memory (DRAM) device, a static random access memory (SRAM) device, or the like. Combinations of the above may also be included within the scope of non-transitory computer-readable storage media. Computer-executable instructions may include, for example, instructions and data that when executed by the processor 214 cause the processor 214 to perform or control performance of a certain operation or group of operations. In some embodiments, the storage 216 may store the data signals generated by the microphone 208, the accelerometer 210, and/or the other sensors 212 and/or data derived therefrom.

The communication interface 218 may include any device or component that facilitates communication with a remote device, such as any of the personal electronic devices 106 of the subject 102, the remote server 110, or any other electronic device. For example, the communication interface 218 may include an RF antenna, an infrared (IR) receiver, a WI-FI chip, a BLUETOOTH chip, a cellular chip, an NFC chip, or any other communication interface.

The battery 220 may include any device or component configured to provide power to the sensor device 200 and/or the components thereof. For example, the battery 220 may include a rechargeable battery, a disposable battery, etc. In some embodiments, the sensor device 200 may include circuitry, electrical wires, etc. to provide power from the battery 220 to the other components of the sensor device 200. In some embodiments, the battery 220 may include sufficient capacity such that the sensor device 200 may operate for days, weeks, or months without having the battery changed or recharged. For example, the sensor device 200 may be configured to operate for at least two months without having the battery 220 charged or replaced.

In some embodiments, the battery 220 may be located in the first lobe 202 and the other components of the sensor device 200 may be in the second lobe 204. The sensor device 200 may include wires or other electrical connections spanning the band 206 such that the electrical power from the battery 220 in the first lobe 202 may be provided to the other components in the second lobe 204.

The communication bus 222 may include any connections, lines, wires, or other components facilitating communication between the various components of the sensor device 200. The communication bus 222 may include one or more hardware components and may communicate using one or more protocols. Additionally or alternatively, the communication bus 222 may include wire connections between the components.

In some embodiments, the sensor device 200 may operate in a similar or comparable manner to the embodiments described in U.S. application Ser. No. 16/118,242, which is hereby incorporated by reference in its entirety.

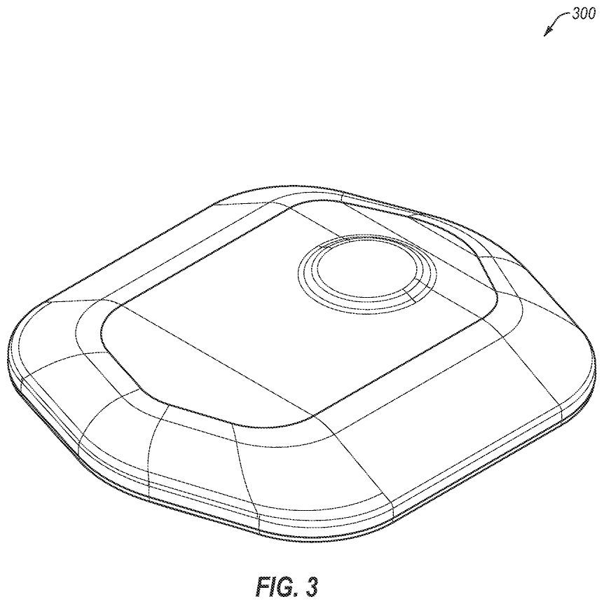

FIG. 3 is a perspective view of another example sensor device 300 that may be implemented in the environment of FIG. 1, arranged in accordance with at least one embodiment described herein. The sensor device 300 may include, be included in, or correspond to any of the sensor devices 104 of FIG. 1, such as the sensor device 104C of FIG. 1. The sensor device 300 may be configured to monitor one or more biological parameters of the subject 102 while disposed, e.g., on the body of the subject 102. For example, the sensor device 200 may generate one or more data signals of one or more biological parameters of the subject 102.

The sensor device 300 may have one or more of the same or similar functional blocks as the sensor device 200 of FIGS. 2A-2B, albeit in a different form factor. For example, the sensor device 300 may include one or more of a microphone, an accelerometer, other sensors, a processor, a storage, a communication interface, a battery, or a communication bus packaged in the generally hexagonal form factor depicted in FIG. 3. In the example of FIG. 3, the battery may be packaged in the same general area as the other components to avoid separating the battery from the other components in different lobes as in the embodiment of FIGS. 2A-2B.

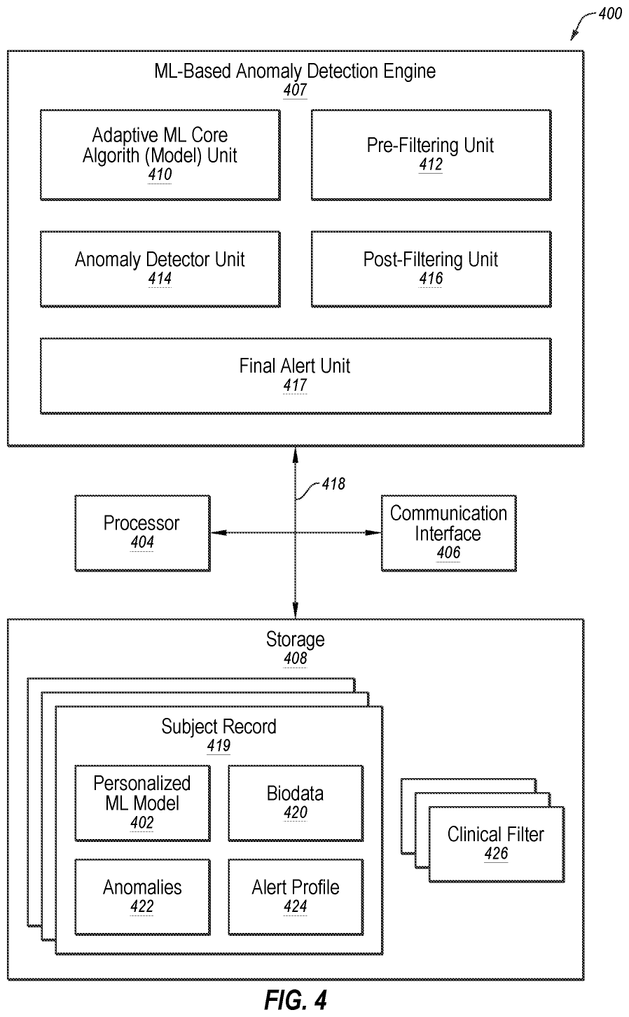

FIG. 4 illustrates an example computing device 400 that may be implemented in the environment of FIG. 1, arranged in accordance with at least one embodiment described herein. The computing device 400 may include, be included in, or correspond to the remote server 110, any of the personal electronic devices 106 of FIG. 1, such as the smartphone 106B, and/or any other device or system. The computing device 400 may be configured to apply AI, ML, and/or other processing to data signals generated by sensors, portions thereof, or data derived from the data signals, to generate and/or update personalized ML models 402 of subjects, detect anomalies in the data signals (or more generally in the biodata), filter detected anomalies, and/or generate alerts.

The computing device 400 may include a processor 404, a communication interface 406, an ML-based anomaly detection engine 407, and a storage 408. The ML-based anomaly detection engine 407 may include an adaptive ML core algorithms (model) unit 410, a pre-filtering unit 412, an anomaly detector unit 414, a post-filtering unit 416, and/or a final alert unit 417. The computing device 400 may further include a communication bus 418.

The processor 404 may include any device or component configured to execute computer instructions to, e.g., apply AI, ML, and/or other processing to data signals generated by sensors, portions thereof, or data derived from the data signals, generate and/or update the personalized ML models 402 of subjects, detect anomalies in the data signals (or more generally in the biodata), filter detected anomalies, and/or generate alerts. In some embodiments, the processor 404 may include an arithmetic logic unit, a microprocessor, a general-purpose controller, or some other processor or array of processors, to perform or control performance of operations as described herein. The processor 404 may be configured to process data signals and may include various computing architectures including a CISC architecture, a RISC architecture, or an architecture implementing a combination of instruction sets. Although illustrated as a single processor 404, multiple processor devices may be included and other processors and physical configurations may be possible. The processor 404 may be configured to process any suitable number format including, but not limited to two's compliment numbers, integers, fixed binary point numbers, and/or floating point numbers, etc. all of which may be signed or unsigned.

The communication interface 406 may include any device or component that facilitates communication with a remote device, such as any of sensor devices 104, 200, 300, the personal electronic devices 106, or any other electronic device that is remote from the computing device 400. For example, the communication interface 406 may include an RF antenna, an IR receiver, a WI-FI chip, a BLUETOOTH chip, a cellular chip, an NFC chip, or any other communication interface. Alternatively or additionally, the communication interface 406 may include or be coupled to one or more plugins, software interfaces, or other interfaces for or with one or more sensors or sensor devices.

The storage 408 may include non-transitory computer-readable storage media or one or more non-transitory computer-readable storage mediums for carrying or having computer-executable instructions or data structures stored thereon. Such non-transitory computer-readable storage media may be any available non-transitory media that may be accessed by a general-purpose or special-purpose computer, such as the processor 404. By way of example such non-transitory computer-readable storage media may include RAM, ROM, EEPROM, flash memory devices (e.g., solid state memory devices), or any other non-transitory storage medium which may be used to carry or store desired program code in the form of computer-executable instructions or data structures and which may be accessed by a general-purpose or special-purpose computer. In some embodiments, the storage 408 may alternatively or additionally include volatile memory, such as a DRAM device, an SRAM device, or the like. Combinations of the above may also be included within the scope of non-transitory computer-readable storage media. Computer-executable instructions may include, for example, instructions and data that when executed by the processor 404 cause the processor 404 to perform or control performance of a certain operation or group of operations. In some embodiments, the storage 408 may store a record 419 for each of one or more subjects, hereinafter “subject record 419”. Each subject record 419 may include a corresponding personalized ML model 402 of the subject, biodata 420 of the subject, anomalies 422 detected in the biodata 420 of the subject, and/or an alert profile 424. Alternatively or additionally, the storage 408 may store one or more clinical filters 426 and/or other data.

The communication bus 418 may include any connections, lines, wires, or other components facilitating communication between the various components of the computing device 400. The communication bus 418 may include one or more hardware components and may communicate using one or more protocols. Additionally or alternatively, the communication bus 418 may include wire connections between the components.

The ML core algorithms (model) unit 410, the pre-filtering unit 412, the anomaly detector unit 414, and the post-filtering unit 416 may each include code such as computer-readable instructions that may be executable by a processor, such as the processor 404, to perform or control performance of one or more methods or operations as described herein.

The ML core algorithms (model) unit 410 may evaluate some or all of the biodata 420 of a given subject and generate or update the subject's personalized ML model 402, e.g., in the subject record 419. In some embodiments, the ML core algorithms (model) unit 410 applies an unsupervised ML algorithm to the biodata 420 of the subject to generate and/or update the personalized ML model 402. For instance, the ML core algorithms (model) unit 410 may generate and/or update the personalized ML model 402 without an annotated set of training data. The ML core algorithms (model) unit 410 may instead generate and/or update the personalized ML model 402 based on unannotated biodata 420 of the subject. Alternatively or additionally, the ML core algorithms (model) unit 410 may apply a supervised ML algorithm to the biodata 420 of the subject to generate and/or update the personalized ML model 402.

In some embodiments, the biodata 420 evaluated by the ML core algorithms (model) unit 410 to generate each personalized ML model 402 may be time-stamped and the ML core algorithms (model) unit 410 may weight the biodata 420 based on its age in the generation or updating of the personalized ML model 402. In some embodiments, the ML core algorithms (model) unit 410 may recognize patterns, trends, baselines, ranges, or the like in the biodata 420 of the subject and generate and/or update the personalized ML model 402 to reflect the pattern, trend, baseline, range, or the like. For example, if the subject has tachycardia and the biodata 420 includes heart rate measurements of the subject, the personalized ML model 402 generated and/or updated by the ML core algorithms (model) unit 410 for the subject may reflect or indicate that the subject has a higher baseline or resting heart rate than a typical population. As another example, if the subject has chronic obstructive pulmonary disease (COPD) and if the biodata 420 includes respiratory rate measurements of the subject, the personalized ML model 402 generated and/or updated by the ML core algorithms (model) unit 410 for the subject may reflect or indicate that the subject has a higher baseline or resting respiratory rate than the typical population.

Some embodiments herein may include or involve automatic anomaly detection in future. This solution may be achieved by a combination of unsupervised and supervised ML learning. In this example, the ML-based anomaly detection is not limited to a subject's personal past historical and current biodata. In some embodiments, unsupervised ML algorithms are used to detect anomaly points in historical personalized biodata. The detected anomaly points may then be applied by supervised ML algorithms as “ground truth” values to estimate the status of anomalies and final alerts in future. For example, embodiments herein may estimate anomalies and alerts related to the subject's health conditions a few hours ahead of time.

The pre filtering unit 412 may be configured to filter the biodata 420 of each subject for biodata that is obviously anomalous and may flag the anomalous biodata to avoid having it evaluated by the ML core algorithms (model) unit 410 in the generation or updating of the subject's personalized ML model 402. For example, if skin temperature measurements in the biodata 420 are 104 degrees Fahrenheit or higher, such as may occur when the subject is in a hot tub or sunbathing, the skin temperature measurements may be flagged so they are not used to generate (or update) an erroneous personalized ML model 402 for the subject.

The anomaly detector unit 414 may be configured to detect anomalies in the biodata 420. In some embodiments, the anomaly detector unit 414 may detect anomalies by comparing new biodata 420 of the subject against the subject's personalized ML model 402, e.g., in the subject record 419. The anomaly detector unit 414 may detect anomalies based on statistics of individual biological parameters and/or based on combinations of all the biological parameters or a subset of two or more of the biological parameters. The anomaly detector unit 414 may flag anomalous data values as anomalies. In some embodiments, the anomalies detected for each subject may be stored in the storage 408 as anomalies 422 in the corresponding subject records 419.

The post-filtering unit 416 may be configured to filter anomalies 422 detected by the anomaly detector unit 414 to determine whether they're indicative of clinical conditions or indicative of the subject being at risk of the clinical conditions. In some embodiments, the post-filtering unit 416 may filter the anomalies 422 according to the clinical filters 426. Each clinical filter 426 may include a set of one or more biological parameter criteria (e.g., a threshold, range, or the like) that, when satisfied by anomalies 422 of a given subject, indicate the given subject has a corresponding clinical condition or is at risk of having the corresponding clinical condition. In some embodiments, one or more of the clinical filters 426 may include multiple criteria that may be satisfied by anomalies in multiple biological parameters where timings of the anomalies have a correlation that satisfies a correlation condition. For example, one of the clinical filters 426 may include first, second, and third criteria that may be satisfied by the detection of first, second, and third anomalies in first, second, and third biological parameters where the timings of the first, second, and third anomalies are substantially identical (e.g., within a predetermined amount of time of each other) (or the timing of one is delayed or advanced by or within or in excess of a predetermined amount of time compared to the others, or the like).

In some embodiments, each clinical filter 426 may be configured to detect a different clinical condition or one or more risk factors of a different clinical condition, such as a respiratory illness, a viral infection, a bacterial infection, a fever, a vaccine reaction, an allergic reaction, a stroke, a mental health disorder, a nervous system disorder, a heart attack, or other clinical condition or risk factors. For example, a clinical filter 426 for detection of fever (hereinafter the “fever clinical filter”) may include physiological criteria relating to multiple biological parameters, including heart rate, respiratory rate, skin temperature, and motion. In more detail, the fever clinical filter may specify detection of anomalies in each of heart rate, respiratory rate, skin temperature, and motion, and more specifically a high heart rate, a high respiratory rate, a high skin temperature, and low motion, all occurring at substantially the same time. The physiological criteria may optionally include a subject-independent physiological criteria. For example, in addition to the detection of subject-dependent anomalies (e.g., anomalies detected relative to the subject's corresponding personalized ML model 402) in each of one or more biological parameters, at least one of the one or more anomalous biological parameters may have to exceed a subject-independent threshold, be beneath a subject-independent threshold, be within a subject-independent particular range, be outside a subject-independent particular range, or the like. As a specific example, the fever clinical filter, in addition to having criteria for high heart rate, high respiratory rate, high skin temperature, and low motion (all of which are subject-dependent), may include a criteria that the heart rate be in excess of 84 beats per minute or other heart rate threshold, or the skin temperature be in excess of 102 degrees Fahrenheit or other temperature threshold, or other subject-independent criteria.

The final alert unit 417 may be configured to generate alerts when the clinical filters 426 are satisfied. Each alert may be entered into a health record of the subject. Alternatively or additionally, each alert may be provided to one or more caregivers of the subject or other individuals as specified in the alert profile 424. The one or more caregivers or other individuals specified in the alert profile 424 may include healthcare workers, relatives of the subject, friends of the subject, or others. The alert profile 424 may in some embodiments specify how to format, deliver, or output the alert (e.g., via email, text, display device, or the like). Alternatively or additionally, the alert profile 424 may specify content of each alert. For example, the alert profile 424 may specify that each alert output at least one of: a name of the clinical condition, a description of the clinical condition, biological parameters included in the biodata 420 of the subject that indicate that the subject has or is at risk of having the clinical condition, or anomalous values of the biological parameters included in the biodata 420 of the subject that indicate that the subject has or is at risk of having the clinical condition.

Thus, according to some embodiments herein, a personalized ML model 402 may be generated for a subject based on biodata 420 specific to that subject. Because anomalies 422 are detected based on the personalized ML model 420 and clinical conditions or risks of clinical conditions are detected (e.g., using clinical filters 426) based on the anomalies 422, some embodiments herein may detect clinical conditions or risk of having a clinical condition where standard procedures based on criteria derived from large populations of subjects may be inadequate.

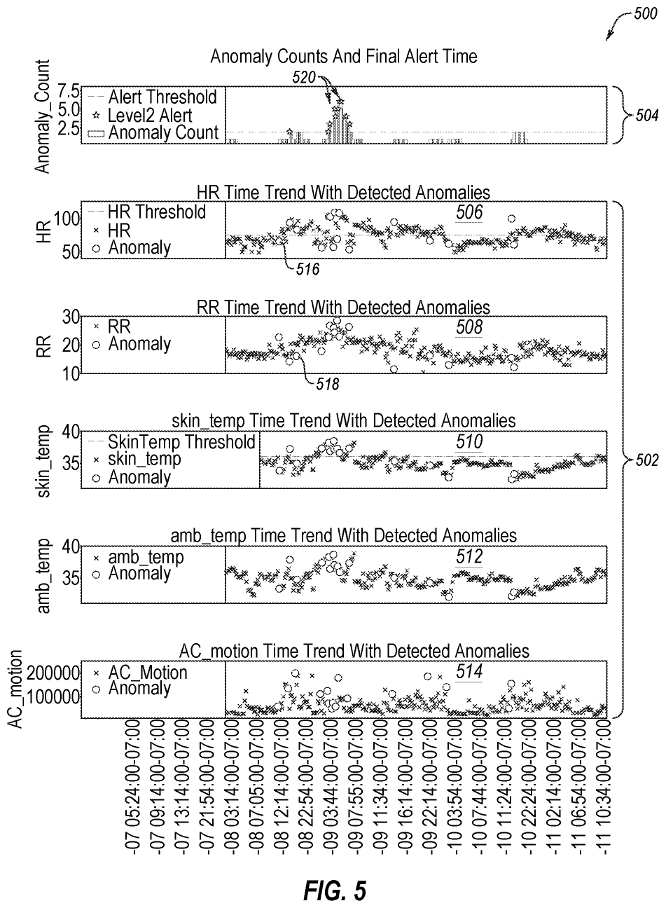

FIG. 5 illustrates experimental results 500 for a subject, arranged in accordance with at least one embodiment described herein. In more detail, the experimental results 500 include biodata 502 and anomaly counts 504 for the subject.

The biodata 502 for the subject includes a time series of measurements 506, 508, 510, 512, 514 for each of the following biological parameters: heart rate (506), respiratory rate (508), skin temperature (510), ambient temperature (512), and motion (514). Anomalies detected in each of the biological parameters are shown as dots or circles, two of which are specifically labeled in FIG. 5 as anomalies 516, 518.

The anomaly counts 504 are shown in rolling time windows.

In the example of FIG. 5, the detected anomalies of the biodata 502 were filtered according to the fever clinical filter mentioned previously and alerts 520 (“Level2 Alert” in FIG. 5) were generated when the fever clinical filter was satisfied by the detected anomalies. A total of eleven alerts 520 were generated in the example of FIG. 5.

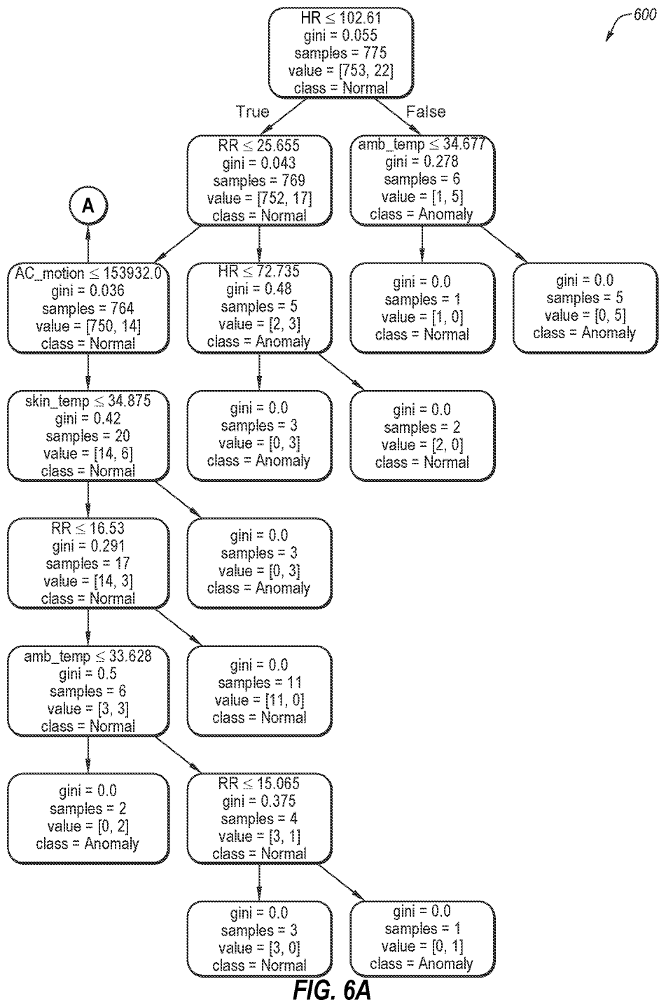

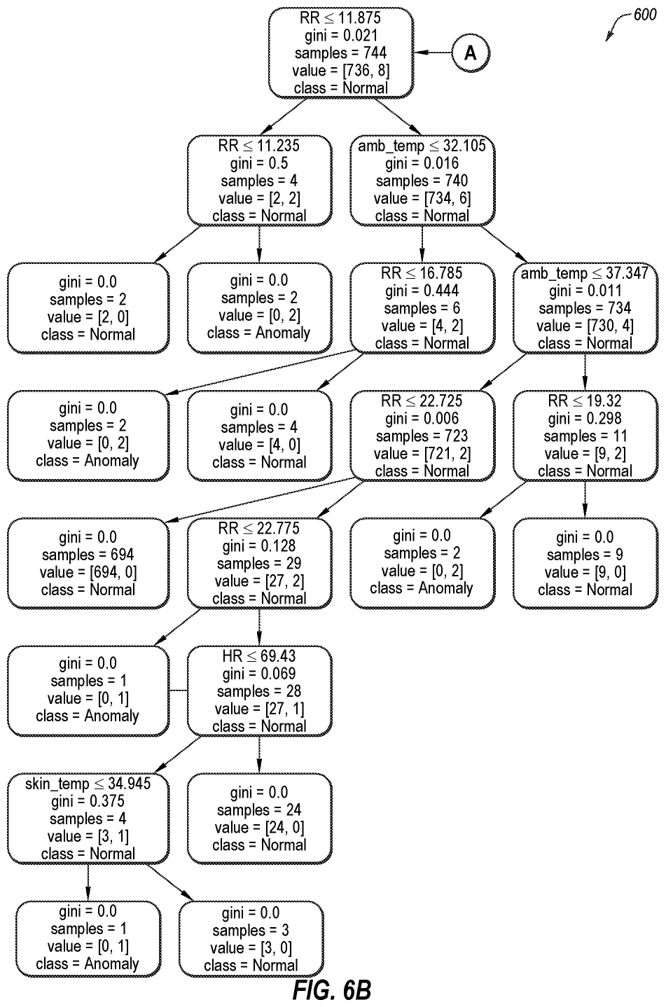

FIGS. 6A and 6B illustrate an example descriptive root-cause analysis graph 600 for the experimental results 500 of FIG. 5, arranged in accordance with at least one embodiment described herein. The root-cause analysis graph 600 may demonstrate relevance or importance of input features and values for each detected anomaly, which may be helpful for disease diagnostics and further actions plan.

After automatic anomaly detection and triggering final alerts, root-cause analysis techniques may be implemented at an end stage of a processing pipeline to provide an easy to understand English interpretation of the results for decision making and designing an action plan. FIGS. 6A and 6B depict the root-cause of detected anomalies and descriptive analysis result for the experimental results of FIG. 5. In this example, the descriptive root-cause analysis graph 600 is a decision tree graph that is a flowchart-like diagram and shows various outcomes from a series of decisions. It can be used as a decision-making tool, for research analysis, or for planning strategy. An advantage of using a decision tree is that it is easy to follow and understand.

Decision trees such as illustrated in FIGS. 6A and 6B have three main parts: a root node, leaf nodes and branches. Each box in FIGS. 6A and 6B represents a node of the tree. The root node is the starting point of the tree. The root node of the descriptive root-cause analysis graph 600 of FIGS. 6A and 6B is the first or top node in FIG. 6A. Every other node in FIGS. 6A and 6B is a leaf node. In the illustrated example, many of the root node and the leaf nodes (apart from those at the extremes) contain questions or criteria to be answered. For example, in the root node, the question or criteria is whether the heart rate (HR) of a subject in question is less than or equal to 102.61 beats per minute (bpm). Branches are the arrows that connect the nodes, showing the flow from question to answer. Each node (apart from those at the extremes) typically has two or more nodes extending from it to other nodes. In the example of the root node in FIG. 6A, if the HR of the subject is less than or equal to 102.61, the answer to the question or criteria is “True” and the flow proceeds from the root node along the left branch. If the HR of the subject is greater than 102.61 bpm, the answer to the question or criteria is “False” and the flow proceeds from the root node along the right branch. For simplicity, the answer for each branch following every node except the root node in FIGS. 6A and 6B that has a question or criteria has been omitted. In practice, the answer for each branch may be included and/or displayed as part of the descriptive root-cause analysis graph 600.

Each node in the descriptive root-cause analysis graph 600 of FIGS. 6A and 6B may include one or more of the following parameters:
A specific input feature (such as HR at the root node in FIG. 6A) and its decision limit to compare. In the example of FIGS. 6A and 6B, the specific input feature is HR, respiratory rate (RR), ambient temperature (amb_temp), motion (AC_motion), or skin temperature (skin_temp).
A “gini” index or Gini impurity is algorithms inside parameters and measures a degree or probability of a particular variable being wrongly classified when it is randomly chosen. It is related to the feature that is selected for a particular node.
“samples” are total remail sample counts at a particular node for classification.
“value [a, b]” indicate the remained normal and anomaly sample counts are a and b respectively at a particular node.
“class” is ultimate identified class at a specific node or leaf.”

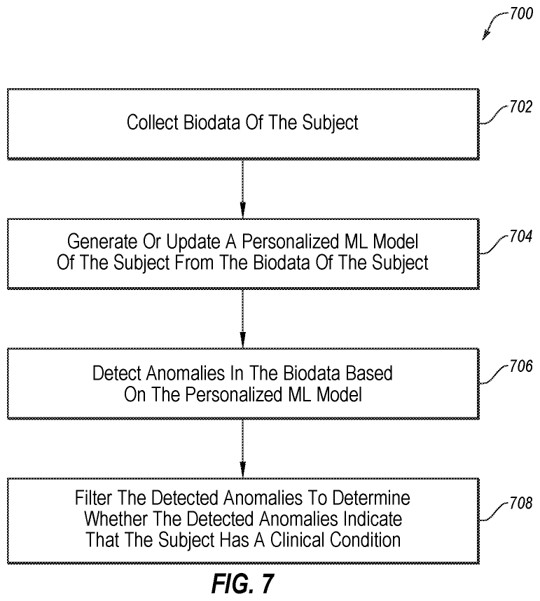

FIG. 7 is a flowchart of a method 700 to detect anomalies, arranged in accordance with at least one embodiment described herein. The method 700 may be programmably performed or controlled by a processor, such as the processor 404, in, e.g., a computing device, such as the computing device 400. In an example implementation, the method 700 may be performed and/or controlled in whole or in part by the remote server 110, or the smartphone 107B (or other personal electronic device) of the subject 102 of FIG. 1. The method 700 may include one or more of blocks 702, 704, 706, and/or 708.

At block 702, the method 700 may include collecting biodata of the subject. For example, biodata 420 may be received, e.g., from one or more of the sensors of FIG. 1. The biodata may be collected before or after a personalized ML model of the subject is generated and may be used to generate or update the personalized ML model (see block 704). In some embodiments, collecting the biodata of the subject at block 702 may include taking or receiving a current time series of measurements for a biological parameter of the subject. Block 702 may be followed by block 704.

At block 704, the method 700 may include generating or updating a personalized ML model of the subject from the biodata of the subject. For example, block 704 may include the ML core algorithms (model) unit 410 of FIG. 4 generating or updating the personalized ML model 402 of the subject from a portion or all of the biodata 420. The personalized ML model of the subject may be generated or updated exclusively from the biodata of the subject. In some embodiments, the personalized ML model of the subject may start as an initial ML model generated using or from population-based data and may then be updated/personalized based on the subject's biodata. Alternatively or additionally, generating the personalized ML model of the subject at block 702 may include a supervised or an unsupervised ML algorithm generating or updating the personalized ML model of the subject. Block 702 may be followed by block 704.

At block 706, the method 700 may include detecting anomalies in the biodata based on the personalized ML model. For example, the anomaly detector unit 414 of FIG. 4 may compare the biodata 420 to the personalized ML model 402, evaluate the biodata 420 in light of the personalized ML model 402, or otherwise process the biodata 420 based on the personalized ML model 402, and flag any anomalies 422 identified in the comparison or evaluation. If the personalized ML model of the subject is updated at block 704, the anomalies may be detected in the biodata before or after the personalized ML model is updated. In some embodiments, the method 700 may additionally include, prior to detecting the anomalies at block 706, the pre-filtering unit 412 pre-filtering the biodata 420 to identify and/or flag any obviously anomalous biodata, e.g., to avoid having the obviously anomalous biodata evaluated by the ML core algorithms (model) unit 410 in the generation or updating of the subject's personalized ML model 402. Block 706 may be followed by block 708.

At block 708, the method 700 may include filtering the detected anomalies to determine whether the detected anomalies indicate that the subject has a clinical condition or is at risk of having the clinical condition. For example, the post-filtering unit 417 may filter the anomalies 422 according to one or more of the clinical filters 426 and may determine that the anomalies 422 indicate that the subject has or is at risk of having a corresponding clinical condition if all the criteria of a given one of the clinical filters 426 is satisfied.

In some embodiments, block 708 includes determining that the detected anomalies indicate that the subject has or is at risk of having the clinical condition. This determination may occur in response to determining that anomalies in multiple different biological parameters included in the biodata of the subject satisfy a filter criterion and that timings of the anomalies have a correlation that satisfies a correlation condition (e.g., all anomalies occurred at the same time or approximately the same time).

In some embodiments, the method 700 may further include, in response to determining that the detected anomalies indicate that the subject has or is at risk of having the clinical condition, generating an alert and entering the alert in a health record of the subject.

In some embodiments, the method 700 may further include, in response to determining that the detected anomalies indicate that the subject has or is at risk of having the clinical condition at block 708, alerting a caregiver that the subject has or is at risk of having the clinical condition. For example, the final alert unit 417 may generate one or more alerts according to the alert profile 424 of the subject. In some embodiments, the caregiver that is alerted may include a healthcare worker, a friend of the subject, a relative of the subject, or other individual.

Alternatively or additionally, in response to determining that the detected anomalies indicate that the subject has or is at risk of having the clinical condition at block 708, the method 700 may further include outputting at least one of: a name of the clinical condition; a description of the clinical condition; biological parameters included in the second biodata of the subject that indicate that the subject has or is at risk of having the clinical condition; anomalous values of the biological parameters included in the second biodata of the subject that indicate that the subject has or is at risk of having the clinical condition; or a descriptive root-cause analysis graph. Outputting such information may facilitate sharing the results with people without data science and machine learning knowledge such as a medical team (e.g., healthcare workers) of the subject.

One skilled in the art will appreciate that, for this and other processes and methods disclosed herein, the functions performed in the processes and methods may be implemented in differing order. Further, the outlined steps and operations are only provided as examples, and some of the steps and operations may be optional, combined into fewer steps and operations, or expanded into additional steps and operations without detracting from the essence of the disclosed embodiments.

Another example method generally corresponding to the method 700 of FIG. 7 may include collecting a first time series of measurements of a first biological parameter of a subject. The method may additionally include collecting a second time series of measurements of a second biological parameter of a subject. The method may additionally include generating or updating a personalized ML model of the subject from the first time series and the second time series. The method may additionally include detecting a first anomaly in the first time series of measurements of the first biological parameter based on the personalized ML model. The method may additionally include detecting a second anomaly in the second time series of measurements of the second biological parameter based on the personalized ML model. The method may additionally include, in response to the first and second anomalies satisfying filter criteria and timings of the first and second anomalies having a correlation that satisfies a correlation condition, determining that the anomalies indicate the subject has or is at risk of having a clinical condition.

Embodiments described herein may be implemented using computer-readable media for carrying or having computer-executable instructions or data structures stored thereon. Such computer-readable media may be any available media that may be accessed by a general-purpose or special-purpose computer By way of example, such computer-readable media may include non-transitory computer-readable storage media including RAM, ROM, EEPROM, CD-ROM or other optical disk storage, magnetic disk storage or other magnetic storage devices, flash memory devices (e.g., solid state memory devices), or any other storage medium which may be used to carry or store desired program code in the form of computer-executable instructions or data structures and which may be accessed by a general-purpose or special-purpose computer. Combinations of the above may also be included within the scope of computer-readable media.

Computer-executable instructions may include, for example, instructions and data which cause a general-purpose computer, special-purpose computer, or special-purpose processing device (e.g., one or more processors) to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Unless specific arrangements described herein are mutually exclusive with one another, the various implementations described herein can be combined to enhance system functionality or to produce complementary functions. Likewise, aspects of the implementations may be implemented in standalone arrangements. Thus, the above description has been given by way of example only and modification in detail may be made within the scope of the present invention.

With respect to the use of substantially any plural or singular terms herein, those having skill in the art can translate from the plural to the singular or from the singular to the plural as is appropriate to the context or application. The various singular/plural permutations may be expressly set forth herein for sake of clarity. A reference to an element in the singular is not intended to mean “one and only one” unless specifically stated, but rather “one or more.” Moreover, nothing disclosed herein is intended to be dedicated to the public regardless of whether such disclosure is explicitly recited in the above description.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Furthermore, in those instances where a convention analogous to “at least one of A, B, and C, etc.” is used, in general, such a construction is intended in the sense one having skill in the art would is used, in general, such a construction is intended in the sense one having skill in the art would understand the convention (e.g., “a system having at least one of A, B, and C” would include but not be limited to systems that include A alone, B alone, C alone, A and B together, A and C together, B and C together, or A, B, and C together, etc.). Also, a phrase presenting two or more alternative terms, whether in the description, claims, or drawings, should be understood to include one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
collecting biodata of a subject;
generating or updating a personalized machine learning (ML) model of a subject from the biodata of the subject;
detecting anomalies in the biodata based on the personalized ML model; and
filtering the detected anomalies to determine whether the detected anomalies indicate that the subject has a clinical condition or is at risk of having the clinical condition.

2. The method of claim 1, wherein generating the personalized ML model of the subject from the biodata comprises generating the personalized ML model of the subject exclusively from the biodata of the subject.

3. The method of claim 1, further comprising, in response to determining that the detected anomalies indicate that the subject has or is at risk of having the clinical condition, generating an alert and entering the alert in a health record of the subject.

4. The method of claim 1, further comprising, in response to determining that the detected anomalies indicate that the subject has or is at risk of having the clinical condition, alerting a caregiver that the subject has or is at risk of having the clinical condition, wherein the caregiver comprises a healthcare worker, a friend of the subject, or a relative of the subject.

5. The method of claim 1, wherein determining that the detected anomalies indicate that the subject has or is at risk of having the clinical condition occurs in response to determining that anomalies in multiple different biological parameters included in the biodata of the subject satisfy a filter criterion and that timings of the anomalies have a correlation that satisfies a correlation condition.

6. The method of claim 1, further comprising, in response to determining that the detected anomalies indicate that the subject has or is at risk of having the clinical condition, outputting at least one of:
a name of the clinical condition;
a description of the clinical condition;
biological parameters included in the biodata of the subject that indicate that the subject has or is at risk of having the clinical condition; or
anomalous values of the biological parameters included in the biodata of the subject that indicate that the subject has or is at risk of having the clinical condition.

7. The method of claim 1, wherein collecting the biodata of the subject comprises taking or receiving a current time series of measurements for at least one biological parameter of the subject.

8. The method of claim 7, wherein the biological parameter of the subject comprises one of heart rate, blood pressure, respiratory rate, skin temperature, heart rate variability, respiratory rate variability, ambient temperature, motion vector values, coughing, sneezing, vomiting, limping, core body temperature, blood oxygenation, blood flow, electrical activity of the heart, electrodermal activity (EDA), ambient sound, EEG brain waves, ambient light level, asthma attack, apnea, hypopnea, arrhythmia, body position, gait, falling, or subject-reported symptom.

9. The method of claim 1, wherein generating or updating the personalized ML model of the subject comprises an unsupervised machine learning algorithm generating or updating the personalized ML model of the subject.

10. The method of claim 1, wherein collecting biodata of the subject comprises collecting data generated by one or more sensors coupled to or in a vicinity of the subject, the one or more sensors including at least one of: a microphone, an accelerometer, a gyrometer sensor, a blood pressure sensor, an optical spectrometer sensor, an electro-chemical sensor, a thermometer, an oxygen saturation sensor, a photoplethysmography (PPG) sensor, an optical sensor, a heart rate sensor, an electrocardiogram (ECG or EKG) sensor, a peripheral oxygen saturation (SpO₂) sensor, a pulse oximeter, an electrodermal activity (EDA) sensor, a brain wave sensor, a light sensor, a gait sensor, or a fall sensor.

11. The method of claim 1, wherein the clinical condition comprises a respiratory illness, a viral infection, a bacterial infection, a fever, a vaccine reaction, an allergic reaction, a stroke, a mental health disorder, a nervous system disorder, or a heart attack.

12. A non-transitory computer-readable storage medium having computer-readable instructions stored thereon that are executable by a processor device to perform or control performance of operations comprising:
collecting biodata of a subject;
generating or updating a personalized machine learning (ML) model of a subject from the biodata of the subject;
detecting anomalies in the biodata based on the personalized ML model; and
filtering the detected anomalies to determine whether the detected anomalies indicate that the subject has a clinical condition or is at risk of having the clinical condition.

13. The non-transitory computer-readable storage medium of claim 12, the operations further comprising, in response to determining that the detected anomalies indicate that the subject has or is at risk of having the clinical condition, generating an alert and entering the alert in a health record of the subject.

14. The non-transitory computer-readable storage medium of claim 12, the operations further comprising, in response to determining that the detected anomalies indicate that the subject has or is at risk of having the clinical condition, alerting a caregiver that the subject has or is at risk of having the clinical condition.

15. The non-transitory computer-readable storage medium of claim 12, wherein determining that the detected anomalies indicate that the subject has or is at risk of having the clinical condition occurs in response to determining that anomalies in multiple different biological parameters included in the biodata of the subject satisfy a filter criteria and that timings of the anomalies have a correlation that satisfies a correlation condition.

16. The non-transitory computer-readable storage medium of claim 12, the operations further comprising, in response to determining that the detected anomalies indicate that the subject has or is at risk of having the clinical condition, outputting at least one of:
a name of the clinical condition;
a description of the clinical condition;
biological parameters included in the biodata of the subject that indicate that the subject has or is at risk of having the clinical condition; or
anomalous values of the biological parameters included in the biodata of the subject that indicate that the subject has or is at risk of having the clinical condition.

17. The non-transitory computer-readable storage medium of claim 12, wherein collecting the biodata of the subject comprises taking or receiving a time series of measurements for at least one biological parameter of the subject.

18. The non-transitory computer-readable storage medium of claim 17, wherein the biological parameter of the subject comprises one of heart rate, blood pressure, respiratory rate, skin temperature, heart rate variability, respiratory rate variability, ambient temperature, motion vector values, coughing, sneezing, vomiting, limping, core body temperature, blood oxygenation, blood flow, electrical activity of the heart, electrodermal activity (EDA), ambient sound, EEG brain waves, ambient light level, asthma attack, apnea, hypopnea, arrhythmia, body position, gait, falling, or subject-reported symptom.

19. The non-transitory computer-readable storage medium of claim 12, wherein generating or updating the personalized ML model of the subject comprises an unsupervised machine learning algorithm generating or updating the personalized ML model of the subject.

20. The non-transitory computer-readable storage medium of claim 12, wherein collecting biodata of the subject comprises collecting data generated by one or more sensors coupled to or in a vicinity of the subject, the one or more sensors including at least one of: a microphone, an accelerometer, a gyrometer sensor, a blood pressure sensor, an optical spectrometer sensor, an electro-chemical sensor, a thermometer, an oxygen saturation sensor, a photoplethysmography (PPG) sensor, an optical sensor, a heart rate sensor, an electrocardiogram (ECG or EKG) sensor, a peripheral oxygen saturation (SpO₂) sensor, a pulse oximeter, an electrodermal activity (EDA) sensor, a brain wave sensor, a light sensor, a gait sensor, or a fall sensor.

21. The non-transitory computer-readable storage medium of claim 12, wherein the clinical condition comprises a respiratory illness, a viral infection, a bacterial infection, a fever, a vaccine reaction, an allergic reaction, a stroke, a mental health disorder, a nervous system disorder, or a heart attack.

22. A method, comprising:
collecting a first time series of measurements of a first biological parameter of a subject;
collecting a second time series of measurements of a second biological parameter of a subject;
generating or updating a personalized machine learning (ML) model of the subject from the first time series and the second time series;
detecting a first anomaly in the first time series of measurements of the first biological parameter based on the personalized ML model;
detecting a second anomaly in the second time series of measurements of the second biological parameter based on the personalized ML model; and
in response to the first and second anomalies satisfying a filter criteria and timings of the first and second anomalies having a correlation that satisfies a correlation condition, determining that the anomalies indicate the subject has a clinical condition.
